(19) United States
(12) Patent Application Publication
(10) Pub. No.: US 2002/0151588 A1
(43) Pub. Date: Oct. 17, 2002

(54) Dissociated glucocorticoid receptor antagonists for the treatment of glucocorticoid associated side effects

(76) Inventors: David S. Thomson, Ridgefield, CT (US); Hans Michael Jennewein, Wiesbaden (DE); Michel Pairet, Stromberg (DE); Frank Kalkbrenner, Ummendorf (DE); Stefan Kreideweiss, Biberach (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/029,720

(22) Filed: Dec. 20, 2001

Related U.S. Application Data
(60) Provisional application No. 60/256,876, filed on Dec. 20, 2000.

(51) Int. Cl. A61K 31/235

(52) U.S. Cl. 514/533

(57) ABSTRACT

The invention relates to the use of glucocorticoid receptor ligands selectively antagonizing the transactivation activity of the glucocorticoid receptor (GR) without affecting the transrepression activity. Compounds having this profile can be used as co-medication with conventional glucocorticoids in the treatment of inflammation and immune diseases. An advantage of this combination therapy is that metabolic side-effects of glucocorticoids are antagonized and only the anti-inflammatory or anti-immune activity of the glucocorticoids is maintained. In such a combination therapy, higher doses of the glucocorticoid can be used leading to better therapeutic efficacy.

### RELATED APPLICATION

The benefit of prior United States provisional application Ser. no. 60/256,876, filed Dec. 20, 2000 is hereby claimed.

### FIELD OF THE INVENTION

The invention relates to the use of glucocorticoid receptor ligands selectively antagonizing the transactivation activity of the glucocorticoid receptor (GR) without affecting the transrepression activity (“dissociated GR antagonists”). Compounds having this profile can be used as co-medication with conventional glucocorticoids in the treatment of inflammation or immune diseases. An advantage of this combination therapy is that a metabolic side-effect of a glucocorticoid is antagonized and only the anti-inflammatory or immunosuppressive activity of the glucocorticoid is maintained. In such a combination therapy, higher doses of the glucocorticoid can be used leading to better therapeutic efficacy.

### BACKGROUND OF THE INVENTION

Glucocorticoids are well known and are frequently used for the treatment of acute and chronic inflammatory diseases, e.g. asthma, rheumatoid arthritis, inflammatory bowel diseases, multiple sclerosis and atopic dermatitis. Despite major efforts to find new targets for anti-inflammatory therapy, glucocorticoids are at present, and will remain in the near future, the most important drugs used for the treatment of inflammatory diseases due to their broad therapeutic spectrum and superior therapeutic effects. Unfortunately, long term systemic as well as local therapies with at least one glucocorticoid is restricted due to its side-effect.

The most common side-effects related with systemic and topical application of a glucocorticoid are metabolic effects, including suppression of HPA axis and the risk of induction of secondary adrenal suppression, induced gluconeogenesis, induced amino acid degradation, changes in electrolyte concentration, changes in lipid metabolism, growth retardation, osteoporosis, skin effects, including impaired wound healing, and skin thinning.

On a molecular level, glucocorticoid receptors (GR) are localized in the cytoplasm of the cell as part of a multi-protein complex composed of GR, heat shock proteins and immunophilins. Binding of glucocorticoids to the GR induces release of the GR from this complex and translocation of the GR to the nucleus.

The ligand-activated receptor dimer activates gene expression by binding to specific DNA sequences (glucocorticoid response elements, GRE) in the promoter regions of glucocorticoid-regulated genes and by interaction with other transactivators and components of the transcription initiation complex (transactivating activity of GR).

The ligand-activated receptor also inhibits transcription of target genes either by binding to negative glucocorticoid response elements (nGRE) or, without binding to the DNA, by direct protein-protein interaction with positively acting transcription factors, e.g. NF-B, AP-1 (transrepressing activity of GR). The GR is able to transrepress target genes in its monomeric form whereas, for DNA-binding, dimerisation or trimerisation of the GR is necessary.

It is assumed that the main mechanism by which a glucocorticoid mediates its anti-inflammatory activity is the transrepression of genes coding for cytokines (e.g. TNF-α, IL-10, IL-6, IL-8 and RANTES), adhesion molecules (e.g. ICAM-1, VCAM) and enzymes (e.g. COX-2) involved in inflammation processes (for recent reviews see Barnes, P J, Clin. Science, 94, 557-572 (1998); Resche-Rigon, M and Gronemeyer, H, Curr. Opin. Chem. Biol., 2, 501-507 (1998); Cato, A C and Wade, E D, Bioessays, 18, 371-8 (1996); Barnes P J and Adcock J, Trends Pharmacol. Sci., 14: 436-441 (1993)) whereas side-effects of glucocorticoids are mainly mediated by GR-DNA-interaction (transactivation).

Thus, it is reasonable to assume that a glucocorticoid inducing only or mainly the transrepressing activity of the GR would display less side-effect.

Because of the known easing and or heeling effect of a glucocorticoid in a variety of medical uses in a subject in need it is desirable to obtain compounds which can reduce a side-effect of a glucocorticoid.

### SUMMARY OF THE INVENTION

The invention relates to the use of glucocorticoid receptor ligands selectively antagonizing the transactivation activity of the glucocorticoid receptor (GR) without affecting the transrepression activity. Compounds having this profile can be used as co-medication with conventional glucocorticoids in the treatment of inflammation and immune diseases. An advantage of this combination therapy is that metabolic side-effects of glucocorticoids are antagonized and only the anti-inflammatory or anti-immune activity of the glucocorticoids is maintained. In such a combination therapy, higher doses of the glucocorticoid can be used leading to better therapeutic efficacy.

### BRIEF DESCRIPTION OF THE DRAWINGS

FIG. 1 shows a screening scheme to identify dissociated GR antagonists.

FIG. 2 shows representative data from test compound EXRS1370SE in GR Competitive Binding Assay. Data points and error bars are the mean and standard deviation of triplicate determinations across three assay plates on a single test occasion.

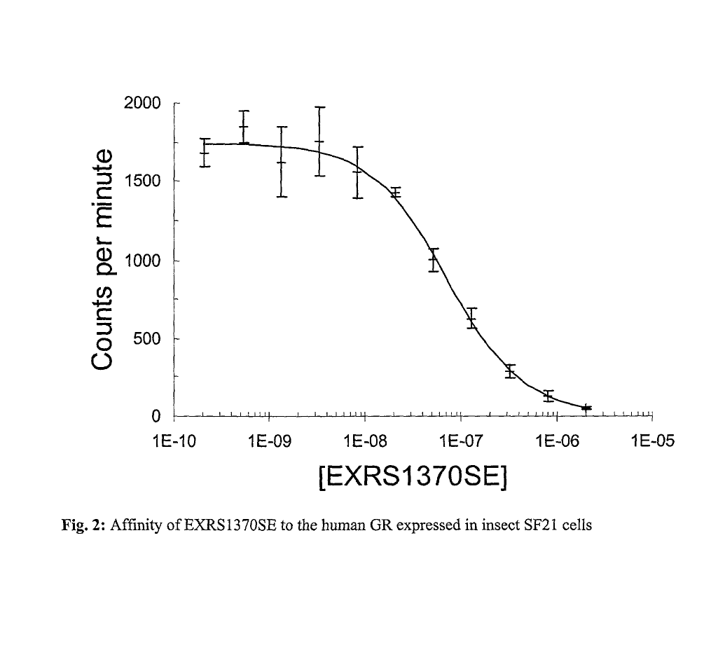

FIG. 3a shows the induction of tyrosine aminotransferase in rat hepatoma cells by EXRS1370SE.

FIG. 3b shows the inhibition of tyrosine aminotransferase pre-stimulated with dexamethasone (3e-9 mol/l) in rat hepatoma cells by R486 and EXRS1370SE.

FIG. 4a shows the induction of the MMTV-promoter in HeLa cells by dexamethasone, prednisolone and EXRS1370SE.

FIG. 4b shows the inhibition of MMTV promoter pre-stimulated with dexamethasone (3e-8 mol/l) in HeLa cells by R486 and EXRS1370SE.

FIG. 5a shows the inhibition of IL-8 induction (1 μg/ml LPS) by dexamethasone and EXRS1370SE.

FIG. 5b shows the inhibition of IL-8 induction (1 tg/ml LPS) by dexamethasone (3e-8 mol/l) and competition with RU486 or EXRS1370SE.

FIG. 6a shows the inhibition of ICAM promoter induction (20 ng/ml TNF-α) by dexamethasone, prednisolone and EXRS 1370SE.

FIG. 6b shows the inhibition of ICAM promoter induction (20 ng/ml TNF-α) by dexamethasone (3e-8 mol/1) and competition with RU486 or EXRS1370SE.

### DESCRIPTION OF THE INVENTION

The present invention provides a method of preventing or suppressing a side-effect associated with glucocorticoid medication of a mammalian, including a human, subject which method comprises administering to the subject as co-medication an effective amount of a glucocorticoid receptor (GR) ligand having antagonist activity, but no agonist activity, in GR-mediated transactivation and no antagonist activity in GR-mediated transrepression of a glucocorticoid sensitive target gene.

An “antagonist” according to the invention can be a substance, which binds to a GR and thereby prevents binding of an endogenous or exogenous agonist.

An “agonist” according to the invention can be an endogenous or exogenous glucocorticoid, which induces by binding to a GR known glucocorticoid mediated cellular effects.

A “partial agonist” according to the invention is a substance binding to a GR and displaying agonistic as well as antagonistic activity.

A “co-medication” according to the invention can be a treatment of a glucocorticoid together with at least one ligand of the invention, which means at the same time with a ligand of the invention or a treatment with a ligand of the invention not at the same time, which can be in advance or after the treatment of the appropriate glucocorticoid.

An effective amount of a ligand of the invention according to the invention is any pharmacologically active amount which is sufficient to reduce at least one side-effect caused by a glucocorticoid treatment without inducing another side-effect more harmful than the side-effect which is to be reduced.

A side-effect according to the invention can be every effect caused by the treatment with a glucocorticoid being unpleasant or negative for a treated subject.

The present invention also provides for a substance which is able to reduce said transacting activity while maintaining said transrepressive activity. Such a substance according to the invention also named “ligand of the invention” is a glucocorticoid receptor (GR) ligand having antagonist activity, but no agonist activity, in GR-mediated transactivation and no antagonist activity in GR-mediated transrepression of a glucocorticoid sensitive target gene. A preferred example of said ligand is {3,5-dibromo-4-[5-isopropyl-4-methoxy-2-(3-methyl-benzoyl-phenoxy]phenyl}-acetic acid.

The aim to use mainly or only the transrepressive activity of the glucocorticoid receptor for therapy can be reached according to the invention by identifying a substance, which selectively inhibits only the transactivation activity of the GR and does not inhibit the transrepression activity (“dissociated GR antagonists”).

In an other embodiment the present invention provides a method to identify substances having the said desired function of a ligand of the invention to reduce a glucocorticoid side-effect.

Such a method according to the present invention can be a method of screening for a dissociated glucocorticoid receptor (GR) antagonist comprising:

a) contacting a candidate substance with a GR;

b) determining binding of the candidate substance to the GR;

c) selecting a candidate substance having binding affinity for the GR;

d) determining activity of the selected candidate substance in GR-mediated transactivation of a glucocorticoid sensitive target gene;

e) selecting a candidate substance having antagonist, but no agonist transactivation activity;

f) determining activity of the selected candidate substance in GR-mediated transrepression of a glucocorticoid sensitive target gene; and

g) selecting the candidate substance having no antagonist activity in transrepression.

A preferred GR-mediated transactiviation according to the invention results in induction of tyrosin aminotransferase (TAT) in e.g. a rat hepatoma cell or for example in stimulation of MMTV (mouse mammary tumor virus) promoter in e.g. a Hela cell.

A preferred GR-mediated transrepression according the invention results e.g in inhibition of a gene having pro-inflammatory or immuno-enhancing activity such as a gene coding for a cytokine or an adhesion molecule or an enzyme each involved in inflammation or in an immune disorder including e.g. an auto-immune disease. For example transrepression can result in inhibition of TNF-α-induced activation of ICAM-1 promoter in a Hela cell or in inhibition of Lipopolysaccharide (LPS)-induced production of interleukin-8 (IL-8) in e.g. a THP1-cell.

As a “glucocorticoid sensitive target gene” of the invention for example can be a gene having pro-inflammatory or immuno-enhancing activity such as a gene coding for a cytokine or an adhesion molecule or an enzyme each involved in inflammation or in an immune disorder including e.g. an auto-immune disease.

The present invention provides a dissociated GR antagonist, i.e. a glucocorticoid receptor (GR) ligand having antagonist activity, but no agonist activity, in GR-mediated transactivation and no antagonist activity in GR-mediated transrepression of a glucocorticoid sensitive target gene which can be found by using the above screening method and displaying the following pharmacological profile:

Displaying

a high affinity to the glucocorticoid receptor and

an antagonistic activity in transactivation of a target promoter by the GR but no agonistic activity (full antagonist in transactivation) and

no antagonistic activity in transrepression of target promoters by the GR.

Said substance of the invention may have or may have no agonistic or partial agonistic activity in transrepression as long as the overall activity does not result in antagonism in transrepression.

A preferred example of said ligand is {3,5-dibromo-4-[5-isopropyl-4-methoxy-2-(3-methyl-benzoyl-phenoxy]phenyl}-acetic acid.

A substance displaying the desired pharmacological profile may then be subjected to an in vivo test by co-administering said substance with a glucocorticoid drug to a subject and determining the capability of the candidate substance to reduce a side-effect preferably a systemic side-effect of the glucocorticoid and to retain the anti-inflammatory activity of the glucocorticoid.

In another aspect, the present invention relates to the use of a dissociated GR antagonist according to the invention, as a co-medication together with at least one glucocorticoid, in the treatment of

an inflammatory disorder or disease or

an immune disorder or disease including an autoimmune disease or

a clinical situation in which treatment with a glucocorticoid is required,

as well as in a disease e.g. as a

Respiratory disease

Rheumatoid disease

Auto-immune disease

Allergy

Vascular disease

Skin disease

Gastrointestinal disease

Renal disease

Liver disease

Ocular disease

Ear disease

Neurological disease

Endocrine disease

Shock

Malignancy

Transplantation

Diabetes and obesity in a mammalian, including a human, subject.

The present invention also relates to a use of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug for the preparation of a pharmaceutical composition for the treatment of an inflammatory disease or an immune disease including an auto-immune disease, in a mammalian, including a human, subject or for the treatment of a said subject in a clinical situation where treatment with a glucocorticoid is required.

And also relates to a use of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug for the preparation of a pharmaceutical composition for the treatment of a

Respiratory disease

Rheumatoid disease

Auto-immune disease

Allergy

Vascular disease

Skin disease

Gastrointestinal disease

Renal disease

Liver disease

Ocular disease

Ear disease

Neurological disease

Endocrine disease

Shock

Malignancy

Transplantation

Diabetes and obesity in a mammalian, including a human, subject.

The present invention relates to a method of treating a mammalian, including a human subject in the need thereof comprising the administration of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug suffering from a condition selected from a

Respiratory disease

Rheumatoid disease

Auto-immune disease

Allergy

Vascular disease

Skin disease

Gastrointestinal disease

Renal disease

Liver disease

Ocular disease

Ear disease

Neurological disease

Endocrine disease

Shock

Malignancy

Transplantation

Diabetes and obesity.

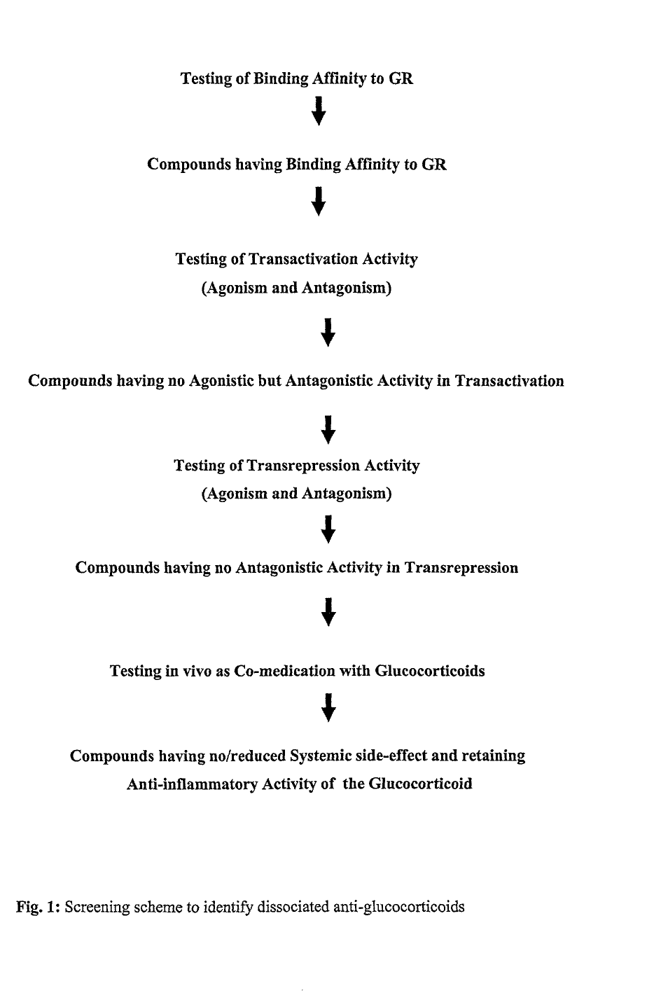

In a further aspect of the present invention a method is provided for preventing or suppressing a side-effect associated with the treatment of inflammatory diseases with a glucocorticoid preferably with a conventional glucocorticoid by using an above identified dissociated GR antagonists according to the invention as co-medication. By using the screening scheme shown in FIG. 1 and described in Example 1, the compound EXRS1370SE (prepared according to WO99/63976) having the following formula

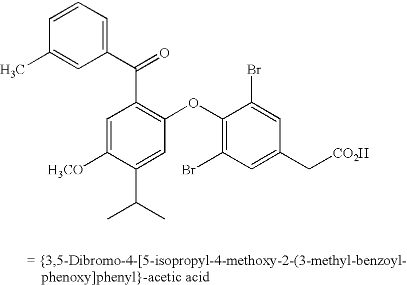

was identified as a compound displaying the desired pharmacological profile (Examples 2-6).

It will be appreciated by a person skilled in the art that the screening method of the present invention is not limited to the specific embodiments of Example 1 but that modifications which the skilled person will be aware of are envisaged as well. For instance, a suspected antagonist or agonist activity of the candidate substance in GR-mediated transactivation may be determined by using any other known glucocorticoid response elements (GRE) and/or genes known to be transactivated by GR and/or other elements involved in transactivation instead of the MMTV (mouse mammary tumor virus) promoter or the endogenous aminotransferase gene in rat hepatoma cells described in the examples herein.

Similarly, for determining activity of the candidate substances in GR-mediated transrepression use may be made of any genes known to be susceptible to such transrepression, in particular genes coding for cytokines, including TNF-α, IL-10, IL-6, IL-8 and RANTES, for adhesion molecules, e.g. ICAM-1, VCAM, and enzymes involved in inflammation processes, e.g. COX-2, and/or of other elements involved in GR-mediated transrepression such as negative glucocorticoid response elements (nGRE) and positively acting transcription factors, e.g. NF-B, AP-1.

Preferably, the method according to the invention is a high throughput screening assay (HTS). HTS relates to an experimental setup wherein a large number of compounds is tested simultaneously. Preferably, said HTS setup may be carried out in microplates, may be partially or fully automated and may be linked to electronic devices such as computers for data storage, analysis, and interpretation using bioinformatics. Preferably, said automation may involve robots capable of handling large numbers of microplates and capable of carrying out several thousand tests per day. Preferably, a test compound which shows a desired inhibitory function in a cell-free system will also be tested in a cell-based system using a cell line according to the present invention. The term HTS also comprises ultra high throughput screening formats (UHTS). Preferably, said UHTS formats may be carried out using 384- or 1536-well microplates, sub-microliter or sub-nanoliter pipettors, improved plate readers and procedures to deal with evaporation. HTS methods are disclosed in U.S. Pat. Nos. 5,876,946A or 5,902,732A herein incorporated in its entirety. The expert in the field can adapt the method described below to a HTS or UHTS format without the need of carrying out an inventive step.

The dissociated GR antagonists identified by the screening method according to the present invention may be used as such or, preferably, in pharmaceutical compositions comprising the same as co-medication in combination with a glucocorticoid drug in the treatment of inflammatory and immune diseases, including autoimmune diseases, and in all clinical situations where treatment with glucocorticoids is required. The glucocorticoid drug of the co-medication may be any glucocorticoid suitable for such treatment including, but not limited to, cortisol, cortisone, corticosterone, dexamethasone, prednisolone etc.

The said dissociated glucocorticoid receptor antagonists will be useful for treating the biological conditions or disorders noted herein in mammalian, and more preferably, in human patients.

The use of said dissociated GR antagonists will prevent or suppress a side-effect associated with conventional systemic and topical glucocorticoid medication.

Such a side-effect include, but are not limited to, a metabolic effect, including suppression of HPA axis and the risk of induction of secondary adrenal suppression, induced gluconeogenesis, induced amino acid degradation, changes in electrolyte concentration, changes in lipid metabolism, growth retardation, osteoporosis, myopathy, hypertension, peptic ulcer, skin effects, including impaired wound healing, and skin thinning.

Examples of a disease and a condition to be treated or to be prevented include, but are not limited to, an acute or a chronic inflammatory disease or an immune disease, including an autoimmune disease or an other clinical situation where treatment with at least one glucocorticoid is required. e.g.

a respiratory disease

a lung disease, e.g. asthma, especially exacerbation of asthma or status asthmaticus, or a form of an obstructive pulmonary disease, especially COPD, or a form of bronchitis, or a form of a restrictive lung disease, especially, allergic alveolitis, or a form of lung edema, especially, toxic lung edema, or sarcoidosis, or granulomatosis etc.

an allergic disease, e.g. hay fever, edema, serum sickness, contact dermatitis, drug reaction, urticaria, bee stings, angioneurotic edema, anaphylaxis etc.

an arthritis, e.g. rheumatoid arthritis, osteoarthritis etc.

an rheumatic carditis,

a rheumatic fever

a connective tissues disease, e.g. systemic sclerosis or systemic lupus erythematosus, dermatomyositis, polymyositis, or mixed connective tissues diseases, polychondritis, Sjögrens syndrome etc.

a vascular disease, e.g. polyarteritis nodosa, granulomatous polyarteritis etc.

a skin diseases, e.g. psoriasis, atopic dermatitis, eczema etc.

a gastrointestinal disease, e.g. an inflammatory bowel disease like chronic ulcerative colitis, Crohn's disease, gastritis, or esophagitis, etc.

a renal disease, e.g. glomerulonephritis, interstitial nephritis

a liver disease, e.g. subacute hepatic necrosis, chronic active hepatitis, alcoholic hepatitis or non-alcoholic hepatitis of various origin like chronic infection with hepatitis B virus or the like, or liver cirrhosis etc.

an occular disease, e.g. keratitis, uveitis, iritis, conjunctivitis, blepharitis, chorioditis, neuritis nervus optici etc.

an ear disease, e.g. otitis externa, otitis media etc.

a cerebral edema, e.g. associated with neoplasms, especially those that are metastatic, or caused by trauma or cerebrovascular accidents etc.

a shock, caused by trauma or associated with an other disease

a neurological disease, e.g. multiple sclerosis, acute encephalomyelitis,

meningitis, myastenia gravis, and various forms of seizure etc.

a malignancy, i.e. acute lymphocytic leukemia, lymphoma, breast cancer, or prostate cancer

an idiopathic thromocytopenia, or haemolytic anemia

an organ transplantation, e.g. suppression of tissue rejection, graft versus host disease etc.

an antiemetic therapy, especially as co-treatment in antiemetic therapy for patients receiving chemotherapy

an endocrine disease, e.g. Thyroiditis, adrenal hyperplasia,

Tendonitis, bursitis

Cushing syndrome

a metabolic disease, e.g. diabetes esp. type 2 diabetes or obesity A pharmaceutical composition of the present invention will comprise a ligand of the invention, i.e. a GR ligand having antagonist activity, but no agonist activity, in GR-mediated transactivation and no antagonistic activity in GR-mediated transrepression, as an active ingredient and may also contain a pharmaceutically acceptable carrier. The pharmaceutical composition may, optionally, also contain a glucocorticoid or another therapeutic ingredient.

The dosage to be administered will vary, i.e. depending of the particular active ingredient used, the age and physical condition of the particular subject, the severity of the conditions to be treated, and the selected route of administration; the appropriate dosage can be readily determined by a person skilled in the art. The dosage to reach a therapeutic effect will range from about 1 μg to about 100,000 μg/kg, preferably about 10 μg to about 30,000 μg/kg and more preferably 10 μg to about 30,000 μg/kg.

A pharmaceutical composition according to the invention may include a composition suitable for oral, rectal, topical and parenteral (including subcutaneous, intramuscular and intravenous) administration, although the most suitable route in any given case will depend on the particular subject and the nature and severity of the condition for which the active ingredient is being administered. The pharmaceutical composition may be conveniently presented in unit dosage form, containing a suitable predetermined amount of the active ingredient(s) and the pharmaceutically acceptable carrier.

General examples of such carriers are water, salt solutions, alcohols, polyethylene glycols, polyhydroxyethoxylated castor oil, gelatine, lactose, starch, amylose, magnesium stearate, talc, silicic acid, fatty acid monoglycerides and diglycerides, pentaerythritol fatty acid esters, microcrystalline cellulose, hydroxymethyl cellulose and polyvinylpyrrolidone.

A composition may be prepared by any of the methods known in the art of pharmacy, generally comprising the steps of uniformly and intimately admixing the active ingredient(s) with liquid carriers or finely divided solid carriers or both and then, if necessary, shaping the product into the desired form of presentation, e.g. by compression or moulding.

Where appropriate, a composition may be in the form of depot compositions or preparations for sustained release.

For oral administration the compositions may be formulated as capsules, troches, wafers, ingestible or buccal tablets, as a powder or granules or as a solution or suspension in an aqueous liquid, a non-aqueous liquid. e.g. an alcohol or an oil, an oil-in-water emulsion or a water-in-oil emulsion, e.g. elixirs and syrups, and the like. The capsules may be, e.g., in the form of sustained release capsules wherein the main capsule contains microcapsules of the active ingredient which release the contents over a period of several hours thereby maintaining a constant level of the drug in the patient's blood.

A pharmaceutical composition suitable for parenteral administration may be prepared as solutions or suspensions of the active ingredients) in water suitably mixed with a surfactant such as hydroxypropylcellulose. Dispersions can also be prepared in glycerol, liquid polyethylene glycols, and mixtures thereof in oils.

The pharmaceutical forms suitable for injection purposes include sterile aqueous solutions or dispersions and sterile powders for the extemporaneous preparation of sterile injectable solutions or dispersions. In all cases, the form must be sterile and must be fluid to the extend that easy syringability exists. It must be stable under conditions of manufacture and storage and must be preserved against the contaminating action of microorganisms, such as bacteria and fungi. The carrier can be a solvent or dispersion medium containing, e.g., water, ethanol, a polyol (such as glycerol, propylene glycol and liquid polyethylene glycol), suitable mixtures thereof, and vegetable oils. The appropriate fluidity can be achieved, e.g. by the use of a coating, such as lecithin, by the maintenance of the required particle size in the case of dispersion and/or by the use of surfactants. For prevention of the action of microorganisms various antibacterial and anti-fungal agents, such as parabens, phenol, sorbic acid, thimerosal and the like, may be included. The incorporation of agents which delay absorption, for example aluminium monostearate and gelatin, into the injectable compositions may also be useful.

Suitable topical formulations include transdermal devices, e.g. plasters, aerosols, creams, ointments, lotions, dusting powders, and the like.

A pharmaceutical composition suitable for rectal administration preferably will be presented as suppositories comprising the active ingredient and a suitable carrier such as cocoa butter.

In addition to the aforementioned carriers a pharmaceutical composition described above may comprise inert diluents, buffers, flavoring agents, binders, such as gum tragacanth, acacia, cornstarch, or gelatin; lubricants, such as magnesium stearate; disintegrating agents, such as corn starch, potato starch, alginic acid and the like; surface active or dispersing agents; granulating agents; thickeners; preservatives, including anti-oxidants; isotonic agents, e.g. sugars or sodium chloride; and the like.

Included herein are exemplified embodiments, which are intended as illustrations of single aspects of the invention. Indeed, various modifications of the invention in addition to those herein will become apparent to those skilled in the art from the foregoing description and drawings. Such modifications are intended to fall within the scope of the present invention.

All publications and patent applications cited herein are incorporated by reference in their entireties.

### EXAMPLE 1

The following screening method was used to identify the dissociated glucocorticoid antagonist compound EXRS1370SE:

In a first step, candidate substances were contacted with the human GR expressed in SF21 insect cells, binding of the candidate substances to the GR was determined as detailed in Example 2 and candidate substances having binding affinity for the GR selected;

In a second step, the selected candidate substances were used to inhibit the GR-mediated transactivation of two glucocorticoid sensitive target genes, namely the luciferase gene operably linked with the glucocorticoid sensitive MMTV (mouse mammary tumor virus) promoter in Hela cells transfected with the construct pHHLuc (Nordeen, S K, Biotechniques, 454-8, 1988) and the endogenous tyrosine aminotransferase gene in rat hepatoma cells; candidate substances having antagonist, but no agonist, activity in GR-mediated transactivation were selected by measuring the expression niveaus of the target genes, for example by conducting a luciferase assay (see Example 4 for further details), in the presence and absence of the candidate substances;

In a third step, the activity of the selected candidate substances in GR-mediated transrepression of two glucocorticoid sensitive target genes, namely the luciferase gene operably linked with the glucocorticoid sensitive ICAM-1 promoter in transfected HeLa cells (Ledebur, H C and Parks, T P, J Biol Chem, 270, 933-43, 1995) and the endogenous IL-8 gene in THP-1 cells, was determined by measuring the expression niveaus of the target genes, for example by ELISA using the “OptEIA human IL-8 set” (Pharmingen, Cat. No. 2654KI) or by conducting a luciferase assay (see Example 5 or 6 for further details), in the presence and absence of the candidate substances and a candidate substance having no transrepression activity, i.e. EXRS1370SE, was selected;

In a fourth step, the candidate substance was subjected to in vivo tests by co-administration with dexamethasone or prednisolone in various inflammation models, e.g. croton-oil induced ear edema, carrageenan-induced paw edema, and measuring a systemic side-effect of the glucocorticoids, e.g. induction of TAT in liver, blood glucose and free fatty acids level, renal function; EXRS1370SE was shown to exhibit no/reduced a systemic side-effect and to retain the anti-inflammatory activity of the glucocorticoid.

### EXAMPLE 2

Affinity of EXRS1370SE to the human GR expressed in SF21 insect cells.

Construction of Recombinant GR Baculovirus

The human GR-alpha gene (GenBank accession number M10901) was amplified by the polymerase chain reaction (PCR) from a plasmid containing full length human GR-alpha cDNA into pCR2.1 vector (Invitrogen). DNA sequencing was carried out on the PCR product to verify that the GR gene sequences are correct. The GR gene was then subcloned into pAcG2T baculovirus transfer vector (Pharmingen) to make the GST-fusion GR-full length (FL) construct pAcG2T-GST-GR-FL. Recombinant baculovirus expressing GST-GR-FL polypeptide was generated by recombination of the construct with linearized baculovirus DNA. Expression of GST-GR-FL polypeptide was carried out by co-inoculation of the recombinant GST-GR-FL baculovirus with three other recombinant baculoviruses expressing human heat-shock proteins hsp90, p23, and hsp70 on SF21 cells. The cells were harvested 72 hours post infection, and cytosolic preparation containing GST-GR-FL polypeptide was carried out as follows.

Cytosolic Preparation of GST-GR-FL Polypeptide

All procedures were performed at 4° C. Cells were washed once with Graces medium, and centrifuged to remove the washing solution. Cells were resuspended in seven volumes of Buffer A over wet cell weight, and applied to a Nitrogen Bomb. The Bomb is sealed and pressured to approximately 700 PSI with stirring. The suspended cell mass/buffer mixture was harvested promptly into the appropriate receiving vessel. The cells mass is then viewed under a microscope to insure all or most of the cells have been ruptured. Although rare, a second pass in the Nitrogen Bomb is sometimes be necessary. Cell debris was removed by centrifugation at 4,000×g for 15 min. The supernatant solution was saved and further ultra-centrifuged at 100,000×g for 75 min. Aliquots of the clarified supernatants were frozen in liquid nitrogen and stored at −80° until use. Buffer A consisted of 20 mM HEPES/Na⁺ pH7.5, 10 mM sodium bisulfite pH7.5, 1 mM DTT, and freshly added 4 μg/mL leupeptin, 4 μg/mL Pepstatin A, 1 mM PMSF, 1 mM sodium molybdate.

GR Competitive Binding Assay

This assay quantitates the ability of test compounds to compete with [6,7-³H(N)]-dexamethasone for binding to recombinant human GR present in an insect cell lysate preparation. The assay buffer was: 10 mM TES, 20 mM Na₂MoO₄.2H₂O, 1.5 mM EDTA, 10% v/v glycerol, 1 mM dithiothreitol, pH=7.4. Test compounds were dissolved to 1 mM in neat DMSO and then further diluted to 10×assay concentration in assay buffer supplemented with 10% v/v DMSO. Compounds were then serially diluted at 10×assay concentrations in 10% DMSO-containing buffer in 96-well polypropylene plates. Binding reaction mixtures were prepared in 96-well Polyfiltronics Unifilter 350 white microtiter filter plates (0.45 m PVDF membrane) by sequential addition of the following assay components: 70 L of GR cocktail containing 45 μL assay buffer and 25 μL cell lysate, 10 μL of 10×test compound solution, and 20 μL of ³H-dexamethasone in assay buffer at 5 nM. Plates were sealed and incubated at 4 C. for 18 to 20 hours followed by addition of 100 μL of 2% w/v dextran-charcoal in assay buffer. After a 5-minute incubation at room temperature, the reaction mixtures were vacuum filtered to remove the charcoal and the filtrates collected in Packard OptiPlates. 150 μL of Microscint 20 was added to each well and after >1 hour at room temperature, the plates were counted in a Packard TopCount plate reader. IC₅₀ values were determined by iterative non-linear curve fitting of the counts per minute data to a 4-parameter logistic equation.

In the assay described above, EXRS1370SE returned an IC₅₀ value of 69 nM after correction for compound determined to be in solution by HPLC analysis.

### EXAMPLE 3

EXRS1370SE does not show agonistic activity in induction of tyrosine aminotransferase (TAT) in rat hepatoma cells but shows antagonistic activity on dexamethasone-induced TAT-induction in these cells.

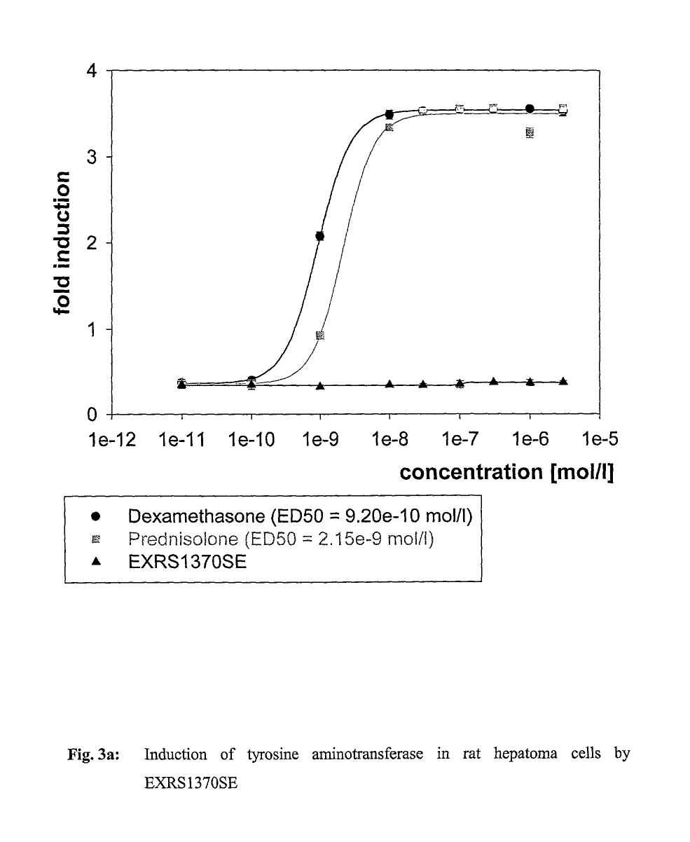

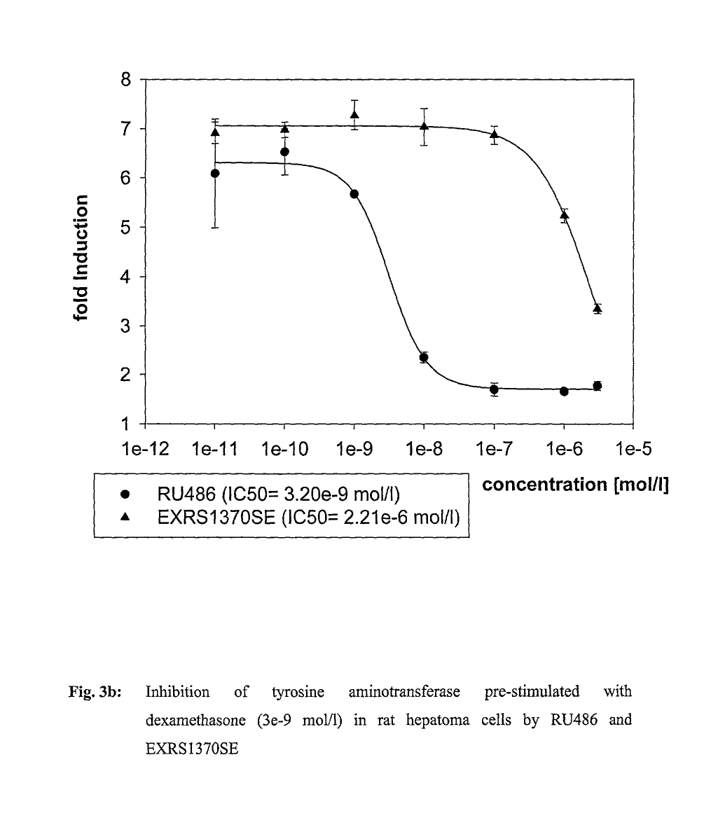

H4-II-E-C3 rat hepatoma cells were incubated overnight in 96 well plates (20,000 cells/200 μl/well) in EMEM medium containing 10% heat inactivated FBS (fetal bovine serum). Next day cells were stimulated with the indicated concentrations of dexamethasone or EXRS1370SE (dissolved in DMSO, final DMSO concentration 0.1%) for 18 hours. Control cells were treated with 0.1% DMSO. After 18 hours, the cells were lysed in a buffer containing 0.1% Triton X-100 and the TAT activity was measured in a photometric assay using tyrosine and -keto g lutarate as substrates (FIG. 3a).

For measuring of the antagonistic activity, the hepatoma cells were pre-stimulated by addition of dexamethasone (3e-9 mol/l) shortly before EXRS1370SE was applied to the cell. The steroidal not dissociated GR/PR antagonist RU486 was used as control (FIG. 3b).

### EXAMPLE 4

EXRS1370SE displayed no agonistic but antagonistic activity in stimulation of MMTV-(mouse mammary tumor virus) promoter in HeLa cells.

HeLa cells stably co-transfected with the pHHLuc-construct containing a fragment of the MMTV-LTR (−200 to +100 relative to the transcription start site) cloned in front of the luciferase gene and the pcDNA3.1 plasmid (Invitrogen) constitutively expressing the resistance for the antibioticum geneticin. Clones with best induction of the MMTV-promoter were selected and used for further experiments.

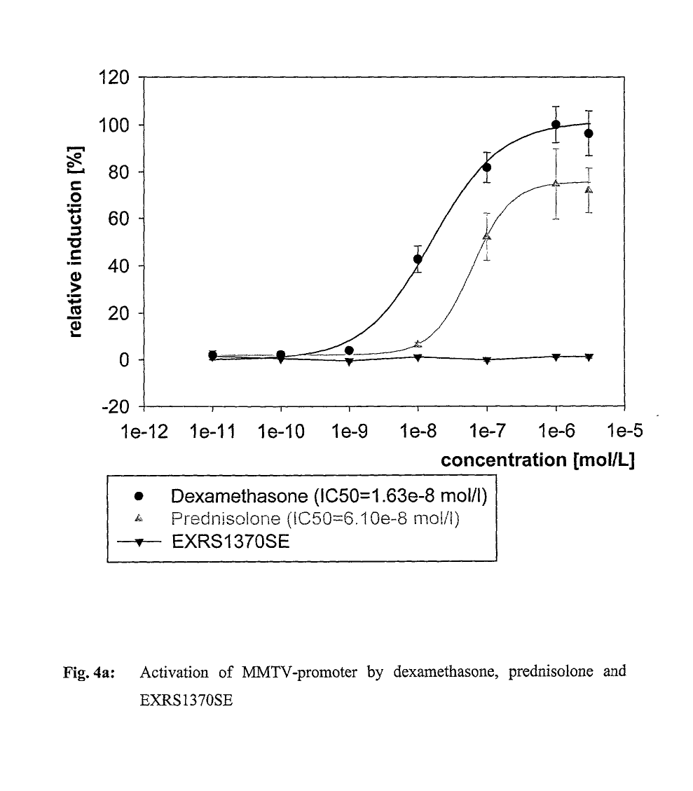

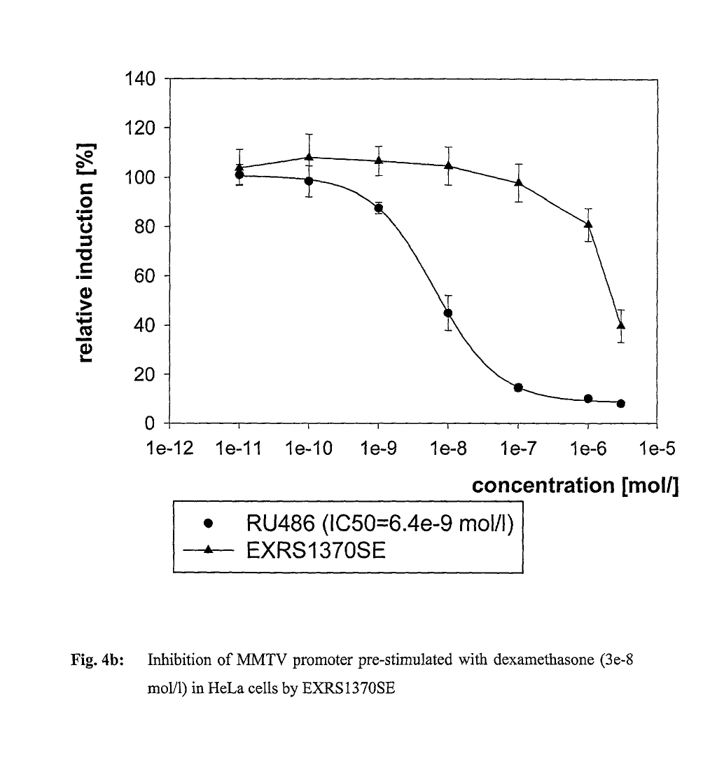

Cells were cultured overnight in DMEM medium w/o phenol red supplemented with 3% CCS (charcoal treated calf serum) and then transferred to 96 well plates (20,000 cells/100 μl/well). Next day the activation of the MMTV-promoter was stimulated by addition of EXRS1370SE or dexamethasone dissolved in DMSO (final concentration 0.1%). Control cells were treated with DMSO only. Twenty four hours later the cells were lysed with cell lysis reagent (Promega, Cat. No. E1531), luciferase assay reagent (Promega, Cat. No. E1501) was added and the flash luminescence was measured using a BMG luminometer (FIG. 4a).

For measurement of antagonistic activity, the MMTV-promoter was pre-stimulated by adding 3e-8 mol/l dexamethasone shortly before EXRS1370SE was applied. The steroidal non-selective GR/PR antagonist RU486 was used as control (FIG. 4b).

### EXAMPLE 5

EXRS1370SE displayed no agonistic and no antagonistic activity in GR-mediated inhibition of LPS-induced IL-8 secretion in U-937 cells.

U-937 cells were incubated for 4 days in RPM11640 medium containing 10% CCS (charcoal treated calf serum). The cells were transferred to 96 well plates (40,000 cells/100 μl/well) and stimulated with 1 μg/ml LPS (dissolved in PBS) in the presence or absence of dexamethasone or EXRS1370SE (dissolved in DMSO). Control cells were treated with 0.1% DMSO.

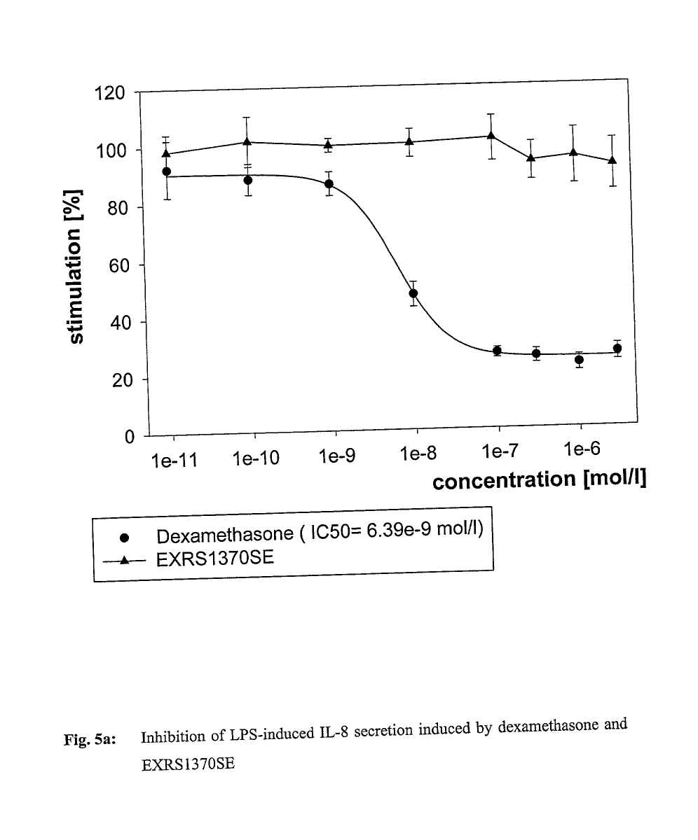

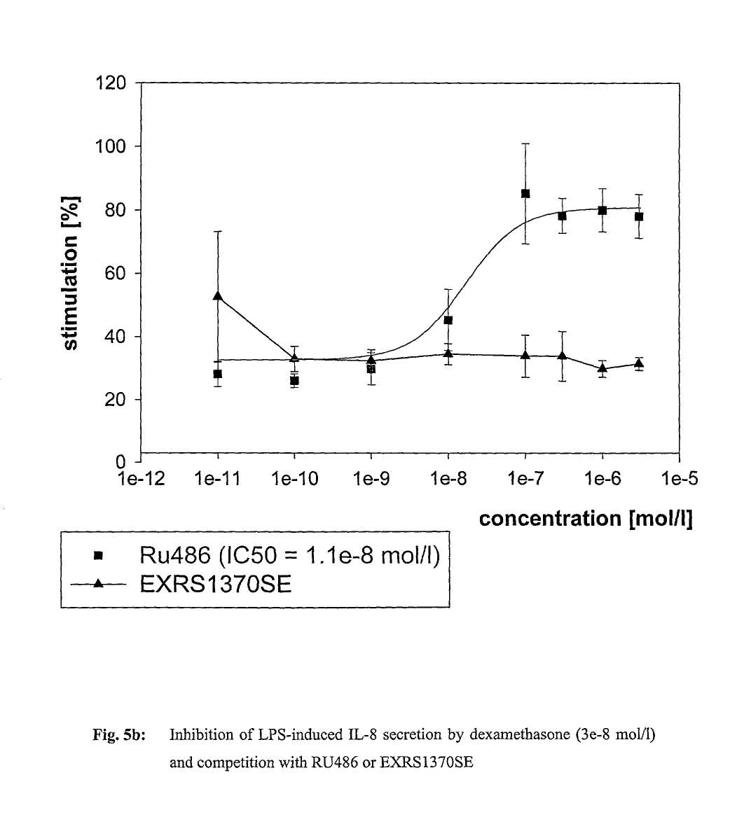

Eighteen hours later the IL-8 concentration in the cell supernatant was measured by ELISA, using the “OptEIA human IL-8 set” (Pharmingen, Cat.No. 2654KI) (FIG. 5a).

For measurement of antagonistic activity, the LPS-induced IL-8 secretion was inhibited by adding 3e-8 mol/l dexamethasone shortly before EXRS1370SE was applied. The steroidal not dissociated GR/PR antagonist RU486 was used as control (FIG. 5b).

### EXAMPLE 6

EXRS1370SE displayed no agonistic and no antagonistic activity in inhibition of TNF-α induced activation of the ICAM-promoter in HeLa cells.

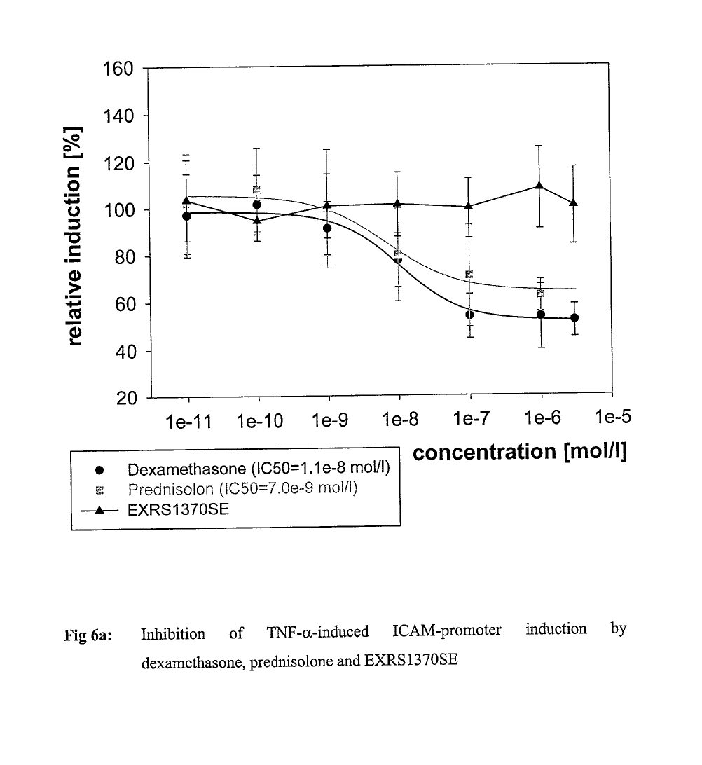

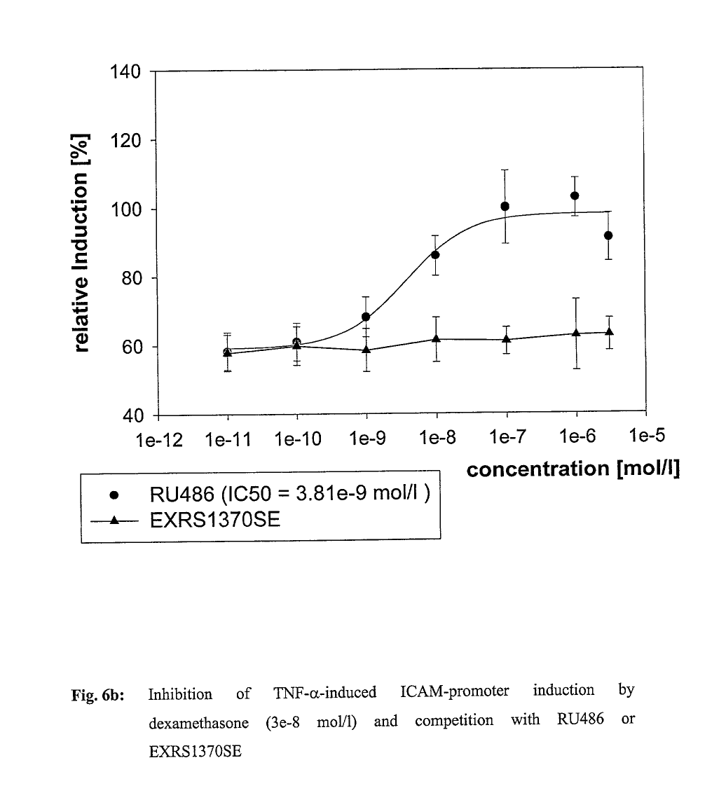

HeLa cells were stably co-transfected with a construct containing a 1.3 kb fragment of the human ICAM-promoter (−1353 to −9 relative to the transcription start site, cloned in front of the luciferase gene and the pcDNA3.1 plasmid (Invitrogen) which constitutively express the resistance for the antibioticum geneticin. Clones with best induction of the ICAM-promoter were selected and used for further experiments. Cells were transferred to 96 well plates (20,000 cells/100 μl/well) in DMEM medium supplemented with 3% CCS. On the following day the activation of the ICAM-promoter was induced by addition of 20 ng/ml recombinant TNF-α (R&D System, Cat. No. 210-TA). Simultaneously the cells were treated with EXRS1370SE or dexamethasone (dissolved in DMSO, final concentration 0.2%). Control cells were treated with DMSO only. Twenty four hours later the cells were lysed with cell lysis reagent (Promega, Cat. No. E1531), luciferase assay reagent (Promega, Cat. No. E1501) was added and flash luminescence was measured using a BMG luminometer (FIG. 6a).

For measurement of antagonistic activity, the TNF-α-induced activation of the ICAM-promoter was inhibited by adding 3e-8 mol/l dexamethasone shortly before EXRS1370SE was applied. The steroidal not dissociated GR/PR antagonist RU486 was used as control (FIG. 6b).

### EXAMPLE 7

EXRS1370XX displays dissociated antagonistic glucocorticoid activity in animal experiments. EXRS1370XX administered in a dose of 100 mg/kg p.o. does not significantly antagonize the anti-inflammatory effect of prednisolone in the ear inflammation model, but can antagonize significantly the prednisolone-induced induction of tyrosine aminotransferase in liver (see Table 1a and b).

Female albino mice (Han:NMRI) obtained form Harlan and weighing about 20-25 g were used. The animals were provided with standardized pellet diet (Altromin 8013) and had tap water freely available. The animals were accommodated in a climate room with a 12-hour light/dark cycle and kept in groups.

EXRS1370XX was synthesized according to WO99/63976, Prednisolone was purchased from a pharmacy as urbasone solubile®. PMA (Phorbol 12-Myristate 13-Acetate), and Mifepristone (11-[4-Dimethylamino]phenyl-17-hydroxy-17[1-propynyl]estra-4,9-dien-3-one; RU-486) were obtained from Sigma (P-8139 and M-8046, respectively).

All test compounds were administered orally (0.2 ml/10 g body weight (bw) ) in a dose of 100 mg/kg p.o. The compounds were “dissolved” in 0.2% hydroxypropylmethylcellulose and olive oil mixed 1:1 (v/v).

Ear Edema

Mice were lightly anaesthetized by ether and 50 ng PMA (5 μl) were applied to each side of the left ear. The right ear remained untreated; solvent alone did not cause any late response. The animals were sacrificed by ether 24 hours later, and a biopsy (diameter 8 mm) was punched out from both ears to assess an increase of neutrophils in the left ear compared with the right ear. Tissue samples were homogenized in 1 ml 0.5% HTAB (Hexadecyl-trimethyl-ammonium-bromide; Sigma H-5882; dissolved in 0.05 M phosphate buffer, pH 6.0) using a tissue homogenizer (IKA-Ultraturrax T5; Janke & Kunkel, Staufen/Breisgau) at 30000 RPM for 15 seconds under cooling. After centrifugation (16000 g, 5 min) the supernatants were frozen until processing for myeloperoxidase (MPO). Determination in the supernatants for MPO, a neutrophil marker enzyme, served as a quantitative index for the neutrophil accumulation. MPO was determined spectrophotometrically at 450 nm using a microplate version of the method of Bradley (1982) and microplate reader (Vₘₐₓ; Molecular Devices, Palo Alto) suitable for kinetic measurements. The values are expressed as increase in milli optical densities per min (m O.D./min) (Table 1a and b).

TAT Induction

Eight hours after compounds were administered the animals were killed and a punch (diameter 8 mm) was taken from the liver and immediately frozen in liquid nitrogen. The weighed liver punches were thawed for 3 minutes and homogenized after the addition 1 ml of phosphate buffer (pH 7.6) using a tissue homogenizer as described above. After centrifugation the supernatants were frozen at −20° C. until processing. For processing the samples were thawed and protein was determined using a kit employing 96 well microtiterplates commercially available from Fa. Pierce (No 23225). The activity of tyrosineaminotransferase (TAT) was determined in the supernatant, which was diluted 1:100 in phosphate buffer. The reaction is started by the addition of tyrosine in pyrodoxal-5-phosphate and α ketoglutaric acid at pH 7.6 and incubated for 30 minutes at 37° C. The reaction is stopped by the addition of 10 M KOH and a further incubation of 30 minutes is allowed for the formation of p-hydroxy-benzylaldehyde, which can be measured at 492 nm using a microplate reader. The values (optical densities) are calculated with respect to the protein concentration (O.D./mg protein) (Table 1a and b).

TABLE 1a
Antagonistic activity of EXRS1370XX on the anti-inflammatory effect of
prednisolone (inhibition of PMA-induced neutrophils influx, measured as
increase in myeloperoxidase, in mice ear) and the metabolic effect of
prednisolone (induction of tyrosine aminotransferase (TAT) in mice liver).
The control in the case of ear inflammation are animals treated with PMA,
the control animals for TAT-induction are treated with vehicle only.
EXRS1370XX did not significantly antagonize the inhibition of ear
inflammation by prednisolone (p = 0.08, Student t test for unpaired
data). However, EXRS1370XX did significantly antagonize the TAT-
induction by prednisolone (p = 0.003, Student t test for unpaired data).

| | | | | Prednisolone |
|---|---|---|---|---|
| | | EXRS1370 | | EXRS1370XX |
| | | XX (100 | Prednisolone | (each 100 |
| | Control | mg/kg) | (100 mg/kg) | mg/kg) |
| Myelo- | 691 ± 161 | 443 ± 90 | 48 ± 13 | 140 ± 47 |
| peroxidase | | | | |
| (treated- | | | | |
| untreated | | | | |
| ear) | | | | |
| [mO.D./ | | | | |
| min] ± | | | | |
| S.E.M. | | | | |
| TAT- | 2.25 ± 0.19 | 3.85 ± 0.43 | 17.75 ± 0.86 | 12.00 ± 1.26 |
| induction | | | | |
| [O.D./mg | | | | |
| protein] ± | | | | |
| S.E.M. | | | | |

TABLE 1b
Antagonistic activity of Mifepristone (RU-486) on the anti-inflamma-
tory effect of prednisolone (inhibition of PMA-induced neutrophils
influx, measured as increase in myeloperoxidase, in mice ear) and the
metabolic effect of prednisolone (induction of tyrosine aminotransferase
(TAT) in mice liver). The control in the case of ear inflammation are
animals treated with PMA, the control animals for TAT-induction are
treated with vehicle only.

| | | | | Prednisolone |
|---|---|---|---|---|
| | | | | Mifepristone |
| | | Mifepristone | Prednisolone | (each 100 |
| | control | (100 mg/kg) | (100 mg/kg) | mg/kg) |
| Myelo- | 623 ± 138 | 931 ± 261 | 34 ± 17 | 1565 ± 435 |
| peroxidase | | | | |
| (treated- | | | | |
| untreated | | | | |
| ear) | | | | |
| [mO.D./ | | | | |
| min ± | | | | |
| S.E.M. | | | | |
| TAT- | 0.81 ± 0.07 | 4.62 ± 0.36 | 13.45 ± 0.93 | 6.60 ± 0.38 |
| induction | | | | |
| [O.D./ | | | | |
| mg pro- | | | | |
| tein] ± | | | | |
| S.E.M. | | | | |

What is claimed is:

1. A glucocorticoid receptor (GR) ligand having antagonist activity, but no agonist activity, in GR-mediated transactivation and no antagonist activity in GR-mediated transrepression of a glucocorticoid sensitive target gene with the proviso that said ligand is not {3,5-dibromo-4-[5-isopropyl-4-methoxy-2-(3-methyl-benzoyl-phenoxy]phenyl}-acetic acid.

2. A glucocorticoid receptor (GR) ligand of claim 1 which has antagonist activity, but no agonist activity, in GR-mediated transactivation and no antagonist activity in GR-mediated transrepression of a glucocorticoid sensitive target gene selected from genes having pro-inflammatory or immuno-enhancing activity, such as a gene coding for a cytokine or an adhesion molecule or an enzyme each involved in inflammation or in an immune disorder including an auto-immune disease.

3. A method of treating a mammalian, including a human, subject in need of glucocorticoid medication which method comprises administering to said subject as co-medication a pharmacologically active amount of a glucocorticoid receptor (GR) ligand having antagonist activity, but no agonist activity, in GR-mediated transactivation and no antagonist activity in GR-mediated transrepression of a glucocorticoid sensitive target gene.

4. A method according to claim 3 in which said ligand is {3,5-dibromo-4-[5-isopropyl-4-methoxy-2-(3-methyl-benzoyl-phenoxy]phenyl}-acetic acid.

5. A method of preventing or suppressing a side-effect associated with glucocorticoid medication of a mammalian, including a human, subject which method comprises administering to the subject as co-medication an effective amount of a glucocorticoid receptor (GR) ligand having antagonist activity, but no agonist activity, in GR-mediated transactivation and no antagonist activity in GR-mediated transrepression of a glucocorticoid sensitive target gene.

6. A method according to claim 5 in which said ligand is {3,5-dibromo-4-[5-isopropyl-4-methoxy-2-(3-methyl-benzoyl-phenoxy]phenyl}-acetic acid.

7. A pharmaceutical composition comprising a glucocorticoid receptor (GR) ligand having antagonist activity, but no agonist activity, in GR-mediated transactivation and no antagonist activity in GR-mediated transrepression of a glucocorticoid sensitive target gene and, optionally, a glucocorticoid.

8. A pharmaceutical composition according to claim 7 in which said ligand is {3,5-dibromo-4-[5-isopropyl-4-methoxy-2-(3-methyl-benzoyl-phenoxy]phenyl}-acetic acid.

9. A use of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug in the treatment of an inflammatory disease or an immune diseases including an auto-immune diseases, in a mammalian, including a human, subject or in the treatment of a said subject in a clinical situation where treatment with a glucocorticoid is required.

10. A use of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug in the treatment of a
Respiratory disease
Rheumatoid disease
Auto-immune disease
Allergy
Vascular disease
Skin disease
Gastrointestinal disease
Renal disease
Liver disease
Ocular disease
Ear disease
Neurological disease
Endocrine disease
Shock
Malignancy
Transplantation
Diabetes and obesity in a mammalian, including a human, subject.

11. A use according to claim 9 or 10 in which said ligand is {3,5-dibromo-4-[5-isopropyl-4-methoxy-2-(3-methyl-benzoyl-phenoxy]phenyl}-acetic acid.

12. A use of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug for the preparation of a pharmaceutical composition for the treatment of an inflammatory disease or an immune disease including an auto-immune disease, in a mammalian, including a human, subject or for the treatment of a said subject in a clinical situation where treatment with a glucocorticoid is required.

13. A use of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug for the preparation of a pharmaceutical composition for the treatment of a
Respiratory disease
Rheumatoid disease
Auto-immune disease
Allergy
Vascular disease
Skin disease
Gastrointestinal disease
Renal disease
Liver disease
Ocular disease
Ear disease
Neurological disease
Endocrine disease
Shock
Malignancy
Transplantation
Diabetes and obesity in a mammalian, including a human, subject.

14. A use according to claim 12 or 13 in which said ligand is {3,5-dibromo-4-[5-isopropyl-4-methoxy-2-(3-methyl-benzoyl-phenoxy]phenyl}-acetic acid.

15. A method of screening for a dissociated glucocorticoid receptor (GR) antagonist comprising:
a) contacting a candidate substance with a GR;
b) determining binding of the candidate substance to the GR;,
c) selecting a candidate substance having binding affinity for the GR;
d) determining activity of the selected candidate substance in GR-mediated transactivation of a glucocorticoid sensitive target gene;
e) selecting a candidate substance having antagonist, but no agonist transactivation activity;
f) determining activity of the selected candidate substance in GR-mediated transrepression of a glucocorticoid sensitive target gene;
and
g) selecting the candidate substance having no antagonist transrepression activity.

16. A method according to claim 15 wherein the GR-mediated transactivation results in induction of tyrosine aminotransferase (TAT) in a rat hepatoma cell or in stimulation of MMTV (mouse mammary tumor virus) promoter in a HeLa cell.

17. A method according to claim 15 or 16 wherein the GR-mediated transrepression results in inhibition of a gene having pro-inflammatory or immuno-enhancing activity, such as a gene coding for a cytokine or an adhesion molecule or an enzyme each involved in inflammation or in a immune disorder including an auto-immune diseases.

18. A method according to claim 17 wherein the GR-mediated transrepression results in inhibition of TNF-α-induced activation of ICAM-1 promoter in a HeLa cell or in inhibition of LPS-induced production of IL-8 in a THP1-cell.

19. A method according to any one of claims 15 to 18 further comprising the step of testing the candidate substance in vivo by co-administering said substance with a glucocorticoid drug to a subject and determining the capability of the candidate substance to reduce a systemic side-effect of the glucocorticoid but retaining the anti-inflammatory activity of the glucocorticoid.

20. A method according to any one of claims 15 to 19 wherein said method is a high-throughput screening assay (HTS).

21. A method of treating a mammalian, including a human subject in the need thereof comprising the administration of a glucocorticoid receptor (GR) ligand having antagonist activity, but no agonist activity, in GR-mediated transactivation and no antagonist activity in GR-mediated transrepression of a glucocorticoid sensitive target gene as co-medication in combination with a glucocorticoid drug suffering from a condition selected from a
Respiratory disease
Rheumatoid disease
Auto-immune disease
Allergy
Vascular disease
Skin disease
Gastrointestinal disease
Renal disease
Liver disease
Ocular disease
Ear disease
Neurological disease
Endocrine disease
Shock
Malignancy
Transplantation
Diabetes and obesity.
